(19) United States
(12) Patent Application Publication
(10) Pub. No.: US 2007/0207993 A1
(43) Pub. Date: Sep. 6, 2007

(54) Molybdenum carbonyl complexes for treating rheumatoid arthritis and other inflammatory diseases

(75) Inventors: Werner E. Haas, Oeiras (PT); Carlos C. Romao, Cascais (PT); Sandra S. Rodrigues, Lisbon (PT); Joao D. Seixas, Odivelas (PT); Ana Rita M. Pina, Lisbon (PT)

Correspondence Address: WILMER CUTLER PICKERING HALE AND DORR LLP, 60 STATE STREET, BOSTON, MA, 02109, US

(73) Assignee: Alfama - Investigacao e Desenvolvimetno De Productos Farmaceuticos Lda, Oeiras (PT)

(21) Appl. No.: 11/642,329

(22) Filed: Dec. 20, 2006

Related U.S. Application Data
(60) Provisional application No. 60/752,571, filed on Dec. 20, 2005.

(51) Int. Cl. A61K 31/555; G01N 33/53

(52) U.S. Cl. 514/184; 435/7.1

(57) ABSTRACT

The present invention relates to molybdenum carbonyl complexes useful for inhibiting tumor necrosis factor (TNF) production and for treating inflammatory diseases.

This application claims priority to U.S. Provisional Application No. 60/752,571, filed Dec. 20, 2005. The entire disclosure of that application is relied upon and incorporated herein by reference.

Throughout this application, various publications are referenced. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure, as it appears in the U.S. Patent and Trademark Office patent file or records, but otherwise reserves any and all copyright rights whatsoever.

### FIELD OF THE INVENTION

The molybdenum carbonyl complexes described herein are useful for inhibiting tumor necrosis factor (TNF) production and for treating inflammatory diseases.

### BACKGROUND OF THE INVENTION

The treatment of acute and chronic inflammatory diseases remains a major challenge. Rheumatoid arthritis is an example of a chronic inflammatory disease for which current treatment is inadequate. The traditional drugs in current use are nonsteroidal anti-inflammatory drugs (NSAIDs), corticosteroids, and various disease-modifying antirheumatic drugs (DMARDs). These drugs are effective only in a subset of patients and their long term use is limited by side effects, some of which are severe.

A major advance in the treatment of rheumatoid arthritis came with the introduction of tumor necrosis factor (“TNF”) antagonists. These drugs, either antibodies or engineered soluble receptors that bind TNF, have improved the treatment of rheumatoid arthritis (1, numbers in parenthesis refer to numbered references at the end of this patent application) and are also useful in a variety of other inflammatory conditions (2-6). A drawback of these DMARDs is that their production is very expensive. Moreover, their long term use is also associated with side effects, some of which are severe (7). However, TNF antagonism is a validated strategy for treating rheumatoid arthritis and other inflammatory conditions (8).

TNF is a pro-inflammatory cytokine produced by a wide spectrum of cells. In excess, TNF may have detrimental systemic effects. The biological effects of TNF depend upon its concentration and site of production. At low concentrations, TNF may produce desirable homeostatic and defense functions such as defending organisms against infectious agents and aiding in recovery from injury. However, at higher concentrations, systemically or in certain tissues, TNF can synergize with other cytokines, notably interleukin-1, to aggravate many inflammatory responses. Because TNF is involved in the pathogenesis of many undesirable inflammatory conditions, means have been sought to inhibit the activity or reduce the production of TNF as a way to control a variety of diseases. Inhibition of TNF can lead to a reduction in inflammatory processes.

Efforts are currently under way to develop small molecular weight TNF inhibitors that can be produced at low cost and that may have fewer side effects by acting locally in inflamed tissues. One strategy to achieve this goal is through the use of endogenously produced, small molecular weight substances that are known to inhibit TNF production. One such molecule is carbon monoxide (CO). CO inhibits TNF production in vitro and in vivo and has shown impressive anti-inflammatory effects in animal models (9, 10). In addition to inhibiting TNF production, CO has additional anti-inflammatory effects. It inhibits the production of other proinflammatory cytokines such as IL-1, IL-6 and MIP-1 (11, 12), enhances IL-10 production (11), inhibits excessive NO production by inducible nitric oxide synthase (13), inhibits mast cell activation (14), and modulates immune responses (15). Exogenous CO may also induce the expression of hemoxygenase-1 (HO-1) either by the transient generation of reactive oxygen species (16) or via the enhancement of IL-10 production (17). HO-1 is known to have a wide variety of protective functions (18), most of which are mediated by its products CO and biliverdin/bilirubin. Thus, the beneficial effects of exogenous CO may be further augmented by the induction of endogenous CO and biliverdin/bilirubin production.

CO inhalation has been a very useful experimental procedure to reveal the beneficial effects of CO in animal disease models. Several patent applications disclose the use of CO as a gas for a wide variety of indications associated with inflammatory reactions (US 2002155166, US 2003039638, US 2003219496, US 2003219497, US 2004052866, WO 03/103585, WO 04/043341). However, CO administration by inhalation is not practical for clinical applications, as it requires special delivery devices such as ventilators, face masks, tents, or portable inhalers. Moreover, CO delivery to therapeutic targets by inhalation is inefficient, because it involves transport of CO by hemoglobin. Hemoglobin binds CO reversibly, but with very high affinity. Therefore, the doses required to deliver CO to therapeutic targets in diseased tissues are likely to be associated with adverse effects.

CO releasing molecules (CORMs) that can deliver CO directly to therapeutic targets without the formation of intermediate CO-hemoglobin complexes have also been developed (19, 20). Impressive, therapeutic effects have been achieved with ruthenium-based CORMs in tissue culture (16), a perfused heart model (20) and in vivo in myocardial infarction models (21). Ruthenium-based CORMs have also been shown to inhibit TNF and excessive NO production in tissue culture (16). A wide variety of CORMs have been disclosed for their use in the treatment of inflammatory diseases and diseases associated with acute or chronic inflammatory reactions (WO 02/092075, WO 04/045598, WO 04/045599, WO 02/078684, US 2004/067261). The potential advantage of CO delivery by CORMs over CO delivery by inhalation is generally recognized. However, CORMs should be able to deliver CO selectively to diseased tissues. The identification of CORMs that are best suited for the treatment of a particular disease remains a major challenge of CORM development. Very little is presently known about chemical reactions of organometallic carbonyl complexes in aqueous solutions.

The present invention is directed to these and other important ends.

### SUMMARY OF THE INVENTION

In one embodiment, methods for inhibiting tumor necrosis factor production in an animal in need thereof are described herein. The methods include administering to the animal an effective amount of a compound of the Formula I:
[Mo(CO)₅Y]Q I
where Y is bromide, chloride or iodide;
Q is [NR₄]⁺, free or complexed with one cyclic polyether molecule or one or more acyclic polyether molecules, or
Na⁺, K⁺, Mg²⁺, Ca²⁺ or Zn²⁺, where each is free or complexed with one cyclic polyether molecule or one or more acyclic polyether molecules,
wherein
each R is independently H or alkyl.

In some embodiments, the one cyclic polyether molecule includes a crown ether from the 18-crown-6 family or the 15-crown-5 family, and the one or more acyclic polyether molecules are of the polyethylene glycol type and of the formula R¹O(CH₂CH₂O)ₙR² where R¹ and R² are each independently H or alkyl, n is greater than or equal to 1, and the acyclic polyethers are within the range of pharmaceutically acceptable polyethylene glycols or mono- or dialkyl polyethylene glycols.

In one embodiment, methods for inhibiting tumor necrosis factor production in a cell are described herein. The methods include contacting the cell with a compound of Formula I.

In one embodiment, methods for treating or preventing a disease in an animal in need thereof are described herein. The methods include administering to the animal an effective amount of a compound of Formula I.

In one embodiment, CO releasing molecules that are useful for the treatment of inflammatory diseases, including without limitation rheumatoid arthritis are described herein.

In one embodiment, a compound of Formula I inhibits the production of TNF. In another embodiment, a compound of Formula I inhibits TNF activity. In yet another embodiment, a compound of Formula I inhibits expression of TNF.

In one embodiment, a method for identifying a compound that inhibits TNF production is described herein as first contacting a test cell with a compound of Formula I:
[Mo(CO)₅Y]Q I

where Y is bromide, chloride or iodide;
Q is [NR₄]⁺, free or complexed with one cyclic polyether molecule or one or more acyclic polyether molecules, or
Na⁺, K⁺, Mg²⁺, Ca²⁺ or Zn²⁺, where each is free or complexed with one cyclic polyether molecule or one or more acyclic polyether molecules,
wherein
each R is independently H or alkyl.
   Then, the level of TNF produced in a test cell sample isolated from the test cell is determined and compared to a level of TNF produced in a control cell sample that has not been contacted with the compound of Formula I. A compound of Formula I that inhibits TNF production is identified when the level of TNF produced in the test cell sample is less than the level of TNF produced in the control cell sample.

In another embodiment, a method for identifying a compound that inhibits TNF production in an animal is described herein as administering an animal a compound of Formula I:
[Mo(CO)₅Y]Q I
where Y is bromide, chloride or iodide;
Q is [NR₄]⁺, free or complexed with one cyclic polyether molecule or one or more acyclic polyether molecules, or
Na⁺, K⁺, Mg²⁺, Ca²⁺ or Zn²⁺, where each is free or complexed with one cyclic polyether molecule or one or more acyclic polyether molecules,
wherein
each R is independently H or alkyl.
   Then, the level of TNF produced in the animal is determined and compared to a level of TNF produced in a control animal that has not been administered the compound of Formula I. A compound of Formula I that inhibits TNF production is identified when the level of TNF produced in the animal is less than the level of TNF produced in the control animal.

### BRIEF DESCRIPTION OF THE FIGURES

FIG. 1 depicts the apparatus used to detect spontaneous CO release from Compound I.1.

FIG. 2 demonstrates the toxicity of Compound I.1 in RAW264.7 cells at 2 hours, 4 hours, and 24 hours using the MTT assay.

FIG. 3 demonstrates CO release in vivo of Compound I.1. Three doses were used and the CO-hemoglobin levels were measured at 0, 30, 120 and, in one case, 330 minutes.

FIG. 4 demonstrates the inhibition of lipopolysaccharide (LPS)-induced TNF production by intraperitoneal application of various doses of Compound I.1.

FIG. 5 demonstrates the inhibition of LPS-induced lethal effects of lipopolysaccharide.

FIGS. 6A-6B demonstrate the average left (FIG. 6A) or right (FIG. 6B) paw volume in an adjuvant arthritis model in rats of the control, positive control (methylene chloride)-treated and Compound I.1-treated groups.

FIGS. 7A-7B demonstrate the average left (FIG. 7A) or right (FIG. 7B) paw circumference in an adjuvant arthritis model in rats of the control, positive control (methylene chloride)-treated and Compound I.1-treated groups.

FIG. 8 demonstrates the arthritis index in an adjuvant arthritis model in rats of the control, positive control (methylene chloride)-treated and Compound I.1-treated groups.

FIG. 9 demonstrates CO release in vivo of Compound I.1 at a concentration of 100 mg/kg. The CO-hemoglobin levels were measured at time intervals.

FIG. 10 demonstrates the in vivo release of CO from Compound I.1 encapsulated in TRIMEB.

### DETAILED DESCRIPTION OF THE INVENTION

In one embodiment, methods for inhibiting tumor necrosis factor production in an animal in need thereof are described herein. The methods include administering to the animal an effective amount of a compound of the Formula I:
[Mo(CO)₅Y]Q I
wherein Y is bromide, chloride or iodide; and
Q is [NR₄]⁺, free or complexed with one cyclic polyether molecule or one or more acyclic polyether molecules, or
Na⁺, K⁺, Mg²⁺, Ca²⁺ or Zn²⁺, where each is free or complexed with one cyclic polyether molecule or one or more acyclic polyether molecules,
wherein
each R is independently H or alkyl.

The cyclic polyether molecule includes, without limitation, crown ethers. In some embodiments, the cyclic polyether includes crown ethers from the 18-crown-6 family or the 15-crown-5 family. The one or more acyclic polyethers are of the polyethylene glycol type and of the formula R¹O(CH₂CH₂O)ₙR² where R¹ and R² are each independently H or alkyl and n is greater than or equal to 1. The acyclic polyether molecules are within the range of pharmaceutically acceptable polyethylene glycols or mono- or dialkyl polyethylene glycols.

When Q is free, Q is not associated with any molecular structure other than a molybdenum complex or molybdenum complexes by electrostatic (ionic) forces. When Q is complexed with one cyclic polyether molecule, or one or more acyclic polyether molecules, these complexed cationic entities are associated with one or more molybdenum anionic complexes by electrostatic bonding. When Q is complexed with acyclic polyethers, an ionic structure results from the interaction between the molybdenum complex or molybdenum complexes and the complexes formed between the acyclic polyethers and the NR₄⁺ or metal cation. The NR₄⁺ or metal cation may accommodate a variable, yet definite and controllable, number of non-covalently bound acyclic polyether molecules giving rise to different polymorphs or solvates. In one embodiment, the NR₄⁺ or metal cation non-covalently binds up to twelve acyclic polyether molecules at one time.

Definitions

As used herein, the term “alkyl” means a C₁-C₁₂ saturated hydrocarbon chain, such as methyl, ethyl, n-propyl, isopropyl, n-butyl, isobutyl, t-butyl, n-pentyl, n-hexyl, n-heptyl, n-octyl, n-nonyl, n-decyl, n-undecyl, or n-dodecyl. In one embodiment, alkyl is a C₁-C₈ or a C₁-C₆ or a C₁-C₄ saturated hydrocarbon chain.

As used herein, the term “animal” includes, without limitation, a human, mouse, rat, guinea pig, dog, cat, horse, cow, pig, monkey, chimpanzee, baboon, or rhesus. In one embodiment, the animal is a mammal. In another embodiment, the animal is a human.

As used herein, the term “halide” means fluoride, chloride, bromide, or iodide.

As used herein, the term “spontaneous release” means release by a thermal, chemical, oxidative, or photodynamic process.

As used herein, the term “release by metabolic process” means release with the involvement of one or more enzymes, such as cytochrome P450 or glutathione S-transferase.

As used herein, the “CO” means carbon monoxide; “CORM” means carbon monoxide releasing molecule; “DMARDS” means disease-modifying antirheumatic drugs; “LPS” means lipopolysaccharide; “n-Bu” means n-butyl; “n—Pr” means n-propyl; “NSAID” means nonsteroidal anti-inflammatory drugs; and “TNF” means tumor necrosis factor.

Compounds of Formula I

In one embodiment, the present compounds of the Formula I are described herein:
[Mo(CO)₅Y]Q I
wherein
Y is bromide, chloride or iodide; and
Q is [NR₄]⁺, free or complexed with one cyclic polyether molecule or one more acyclic polyether molecules, or
Na⁺, K⁺, Mg²⁺, Ca²⁺ or Zn²⁺, where each is free or complexed with one cyclic polyether molecule or one or more acyclic polyether molecules,
wherein
each R is independently H or alkyl.

The cyclic polyether molecule includes, without limitation, crown ethers. In some embodiments, the cyclic polyether includes crown ethers from the 18-crown-6 family or the 15-crown-5 family. The one or more acyclic polyethers are of the polyethylene glycol type and of the formula R¹O(CH₂CH₂O)ₙR² where R¹ and R¹ are each independently H or alkyl and n is greater than or equal to 1. The acyclic polyether molecules are within the range of pharmaceutically acceptable polyethylene glycols or mono- or dialkyl polyethylene glycols.

The compounds of Formula I provide convenient stability under air at room temperature to allow easy manipulation. Moreover, the compounds of Formula I provide the advantage of improved stability in water, including under the acidic pH range found, for example, in the gastric fluid. Without wishing to be bound by theory, applicants believe that this stability derives from the lower basicity of the halide anion relative to other alternative uninegative substituents Y, e.g., hydrocarbyl anions (R⁻), RO⁻, RS⁻, RCOO⁻.

The compounds of Formula I bearing a tetraalkylammonium cation also provide improved stability in water at physiologic pH relative to their analogues with alkaline cations, even when such an alkaline cation is stabilized by a cyclic or acyclic chelating polyether. Again without wishing to be bound by theory, applicants believe that this stability in water derives at least in part from the favorable cation-anion interaction provided by a tetraalkylammonium cation.

In addition, the compounds of Formula I provide enhanced release of carbon monoxide, for example, in response to attack by radical oxygen species, relative to thermally induced carbon monoxide release (substitution) in the absence of such species. Because the onset of this release is very facile, the compounds of Formula I also provide efficient release of carbon monoxide at an inflammatory site in an animal where radical oxygen species can be generated or accumulated in biologically elevated concentrations.

In some embodiments, Y is bromide or chloride.

In other embodiments, in a compound of Formula I, Y is bromide.

In still other embodiments, Y is iodide.

In some embodiments, Q is a tetraalkylammonium cation.

In further embodiments, Q is a tetraethylammonium cation, a tetra(n-butyl)ammonium cation, a tetra(n-propyl)ammonium cation, a tetra(i-propyl)ammonium cation or a tetramethylammonium cation.

In other embodiments, Q is a tetraethylammonium cation.

In some embodiments, R is (C₁-C₁₂)-alkyl. In other embodiments, R is (C₁-C₈)-alkyl. In further embodiments, R is (C₁-C₆)-alkyl. In yet other embodiments, R is (C₁-C₄)-alkyl.

In some embodiments, Q is [NH₄]⁺, Na⁺, K⁺, Mg²⁺, Ca²⁺, or Zn²⁺.

In some embodiments, Q is complexed with one cyclic polyether molecule or one or more acyclic polyether molecules. In some embodiments, the one cyclic polyether molecule includes crown ethers from the 18-crown-6 family or the 15-crown-5 family. In other embodiments Q is complexed by one or more acyclic polyethers in a coordination sphere comprising from four to twelve oxygen atoms of the ethyleneglycol or polyethylene glycol type chains. In yet another embodiment, Q is complexed by six, eight, or twelve acyclic polyether molecules. In another embodiment, Q is complexed by three acyclic diethers. In further embodiments, Q is complexed with one, two, or three polyether molecules.

In some embodiments, Q is complexed with more than one acyclic polyether molecules of the formula R¹O(CH₂CH₂O)ₙR² where R¹ and R² are each independently H or alkyl, n is greater than or equal to 1, and the polyether molecules are within the range of pharmaceutically acceptable polyethylene glycols or mono- or dialkyl polyethylene glycols. In further embodiments, when Q is complexed with more than one ether of the formula R¹O(CH₂CH₂O)ₙR², each R¹ and R² of each polyether molecule is independently H or alkyl, so that each polyether of the formula R¹O(CH₂CH₂O)ₙR² may be different and each R¹ or R² may be different than an R¹ or R² in another polyether molecule.

In some embodiments, the crown ether is selected from the 18-crown-6 family or the 15-crown-5 family.

In some embodiments, the polyether molecule is of the formula R¹O(CH₂CH₂O)ₙR² where R¹ and R² are each independently H or alkyl, n is greater than or equal to 1, and the polyether molecules are within the range of pharmaceutically acceptable polyethylene glycols or mono- or dialkyl polyethylene glycols. In some embodiments, n ranges from 1 to 200, or from 1 to 100, or from 1 to 75, or from 1 to 50, or from 1 to 25. In further embodiments, n is not more than 200. In still further embodiments, n is not more than 100. In yet further embodiments, n is not more than 75, or not more than 50, or not more than 25.

In further embodiments, specific acyclic ethers include, without limitation, monoglyme, diglyme, triglyme, PEG 400, PEG 1000, PEG 2000, PEG 3000 and PEG 4000, and methylPEG400.

In some embodiments, Q is [NH₄]⁺, Na⁺, K⁺, Mg²⁺, Ca²⁺, or Zn²⁺ complexed with a cyclic polyether molecule from the 18-crown-6 family or the 15-crown-5 family.

In some embodiments, the cyclic crown ether is 18-crown-6,15-crown-5, dibenzo[1,8]-crown-6, or dicyclohexyl[1,8]-crown-6.

In other embodiments, Q is K⁺ or NH₄⁺ complexed with a cyclic polyether molecule from the 18-crown-6 family.

In further embodiments, Q is K⁺ or NH₄⁺ complexed with dibenzo[18]-crown-6 or dicyclohexyl[18]-crown-6.

In some embodiments, Q is Na⁺ complexed with a polyether molecule from the 15-crown-6 family.

In further embodiments, Q is Na⁺ complexed with [15]-crown-5.

In some embodiments, Q is [NH₄]⁺, Na⁺, K⁺, Mg²⁺, Ca²⁺, or Zn²⁺ complexed with at least one polyether molecule of the formula R¹O(CH₂CH₂O)ₙR² where n is greater than or equal to 1 and where each R¹ and R² is each independently H or alkyl so that each polyether molecule is the same or different. The polyether molecules are within the range of pharmaceutically acceptable polyethylene glycols. When the compound of Formula I is complexed with more than one polyether molecule, the polyether molecules can be the same or different.

In some embodiments, the polyether molecule is monoglyme, diglyme, triglyme, PEG 400, PEG 1000, PEG 2000, PEG 3000 and PEG 4000, or methylPEG400.

In yet other embodiments, the polyether molecule is diglyme.

In other embodiments, Q is K⁺ or [NH₄]⁺ and is complexed with at least one polyether molecule of the R¹O(CH₂CH₂O)ₙR² where n is greater than or equal to 1, each R¹ and R² is each independently H or alkyl, and the polyether molecules are within the range of pharmaceutically acceptable polyethylene glycols and wherein each polyether molecule is the same or different.

In further embodiments, Q is K⁺ or [NH₄]⁺ and is complexed with three diglymes.

In one embodiment, the compound of Formula I is one of the following compounds:

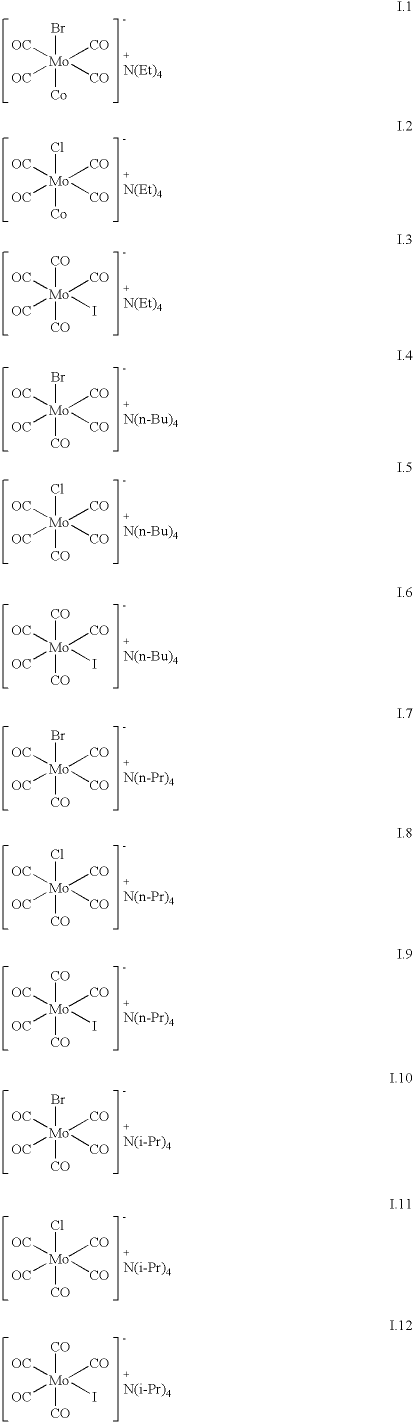

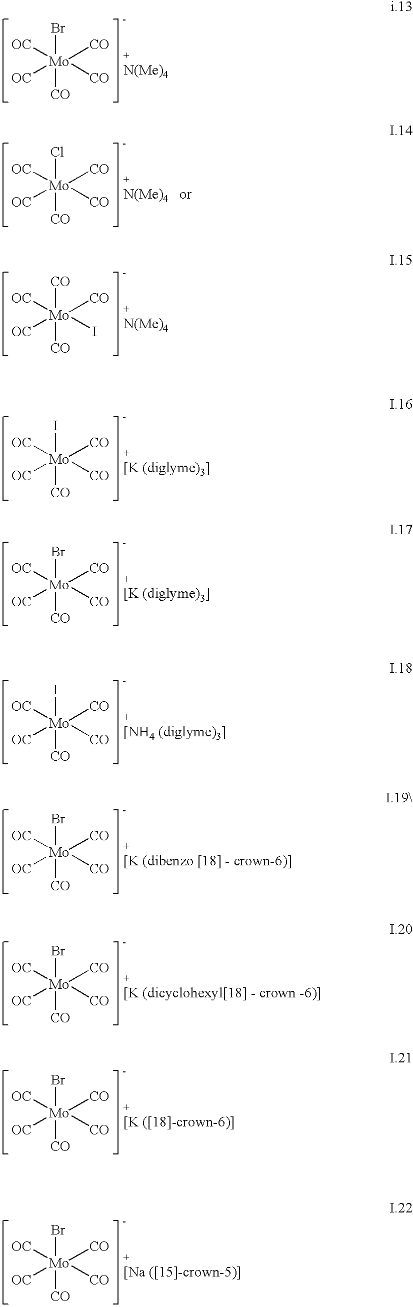

In another embodiment, the compound of Formula I is one of the following compounds:

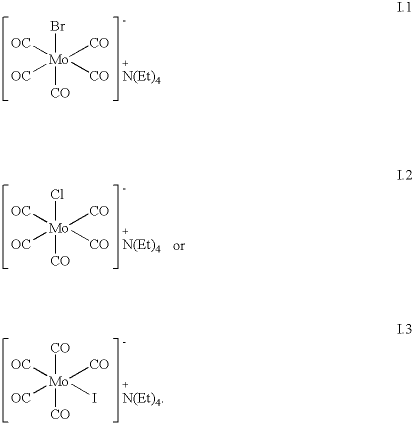

In another embodiment, the compound of Formula I is 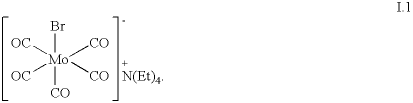
Methods of Making Compounds of Formula I

The compounds described herein can be prepared using a variety of methods well known in the art of molybdenum organometallic chemistry. The common starting material is Mo(CO)₆ that is commercially available or accessible from other Mo salts through known procedures. Tetralkylammonium halides are usually commercially available or can be prepared by alkylation of the corresponding amines, which are also commercially available. General synthetic routes to many of the compounds described herein are known in the art of molybdenum organometallic chemistry as follows.

For example, the iodide [Mo(CO)₅I][K[diglyme)₃] was first reported in 1959 (22, 23).

The introduction of tetralkylammonium counter ions (27) led to the stabilization of these complexes in the solid state allowing for the complete series of complexes [Mo(CO)₅X][NR⁴] to be prepared and characterized (X=Cl, Br, I). Cr and W cogeners of the fluoride analogue, [Mo(CO)₅F]⁻ have been prepared by use of KF and crown-ethers (24, 25).

A slight modification of Abel's method, reported in 1985 (26), using more accessible solvents and lower temperatures, was found appropriate for the preparation of compounds of Formula I. This method consists of refluxing mixtures of Mo(CO)O₆ and the appropriate tetraalkylammonium halide (X is Cl, Br, or I; R is alkyl) in THF and precipitating the compounds by sequential cooling and addition of diethyl ether as depicted in equation (1).

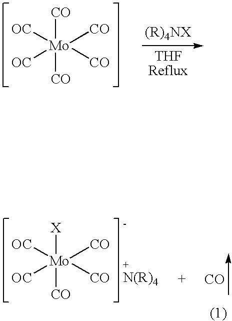

This preparation resulted in high yields (approximately 90-95%).

Compounds of Formula I can also be prepared via halide replacement of photochemically generated [Mo(CO)₅L] complexes with labile ligands (e.g., L=Me₃N, NCMe, THF, Et₂S).

Compound of Formula I can also be prepared according to equation (2) for cations with acyclic polyether molecules. This method consists of heating Mo(CO)₆ and the appropriate metal halide (M is [NH₄]⁺, Na⁺, K⁺, Mg²⁺, Ca²⁺, or Zn²⁺; X is Cl, Br, or I) in the appropriate ether. In some embodiments, the reaction is heated for 1 to 2 hours. The suspension should be filtered hot and hexane added to the cooled filtrate. Diglyme is used in the equation and is representative of other acyclic polyether molecules. Tetraalkylammonium compounds complexed to one or more polyethers are prepared by methods known to those of skill in the art, including methods similar to those described herein.

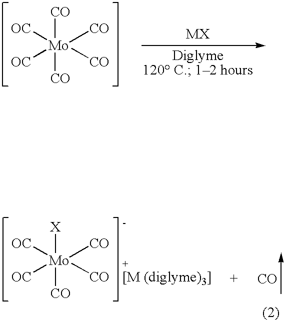

Compounds of Formula I with crown ethers are made in a similar manner to the reaction given in equation (1). This method consists of refluxing mixtures of Mo(CO)₆ and the appropriate metal halide (M is [NH₄]⁺, Na⁺, K⁺, Mg²⁺, Ca²⁺, or Zn²⁺; X is Cl, Br, I) in the appropriate crown ether according to the size of the metal cation. The solution is filtered and hexane is added to the cooled filtrate to precipitate the final compound. Tetraalkylammonium compounds complexed to cyclic polyethers are prepared by methods known to those of skill in the art, including methods similar to those described herein. 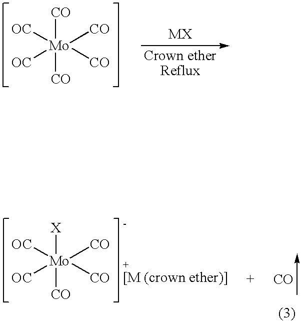
Therapeutic Uses of the Compounds of Formula I

In one embodiment, a compound of Formula I exhibits a therapeutic effect in whole or in part due to the generation of free carbon monoxide. Carbon monoxide can be released from a compound of Formula I either by a spontaneous process or by a metabolic process, i.e., with the involvement of one or more enzymes. The release of CO from the compound is in some embodiments assisted by donor molecules within an animal, such as water, proteins, or nucleotides.

In one embodiment, the compounds of Formula I release CO at specific sites in an animal, such as inflamed tissues or pre-atherosclerotic lesions of an artery. In another embodiment, the compounds of Formula I preferentially release CO in the presence of a reactive oxygen species that is generated at an inflammatory site or in an atherosclerotic lesion.

In one embodiment, compounds of Formula I are TNF inhibitors. In another embodiment, Compound I.1 is a TNF inhibitor. In one embodiment, compounds of Formula I are useful for the treatment of a disease known or suspected to be initiated or promoted by TNF, and are useful for the treatment of inflammatory diseases.

Treatment or Prevention of Inflammatory Diseases

The compounds of Formula I can be used to treat or prevent an inflammatory disease. Inflammatory diseases can arise where there is an inflammation of the body tissue. Examples of inflammatory diseases treatable or preventable using the compounds of Formula I, include, but are not limited to, transplant rejection; chronic inflammatory disorders of the joints, such as arthritis, rheumatoid arthritis, osteoarthritis and bone diseases associated with increased bone resorption; inflammatory bowel diseases such as ileitis, ulcerative colitis, Barrett's syndrome, and Crohn's disease; inflammatory lung disorders such as asthma, adult respiratory distress syndrome (ARDS), and chronic obstructive airway disease; inflammatory disorders of the eye such as corneal dystrophy, trachoma, onchocerciasis, uveitis, sympathetic ophthalmitis and endophthalmitis; chronic inflammatory disorders of the gum, such as gingivitis and periodontitis; tuberculosis; leprosy; inflammatory diseases of the kidney such as uremic complications, glomerulonephritis and nephrosis; inflammatory disorders of the skin such as sclerodermatitis, psoriasis and eczema; inflammatory diseases of the central nervous system, such as chronic demyelinating diseases of the nervous system, multiple sclerosis, AIDS-related neurodegeneration and Alzheimer's disease, infectious meningitis, encephalomyelitis, Parkinson's disease, Huntington's disease, amyotrophic lateral sclerosis and viral or autoimmune encephalitis; autoimmune diseases such as diabetes mellitus, immune-complex vasculitis, systemic lupus erythematosus (SLE); inflammatory diseases of the heart such as cardiomyopathy, ischemic heart disease hypercholesterolemia, and atherosclerosis; as well as inflammation resulting from various diseases such as preeclampsia, chronic liver failure, brain and spinal cord trauma, and cancer. The compounds of Formula I can also be used to treat or prevent the progression of an inflammatory disease and/or to reduce the symptoms of the inflammatory disease. In one embodiment, the compounds of Formula I are useful for treating or preventing pain associated with an inflammatory disease.

The inflammatory disease treatable or preventable by administration of an effective amount of a compound of Formula I can also be a systemic inflammation of the body. Examples of systemic inflammation include but are not limited to, gram positive or gram negative shock, sepsis, septic shock, hemorrhagic or anaphylacetic shock, or SIRS.

In one embodiment, the inflammatory disease is circulatory shock, sepsis, systemic inflammatory response syndrome, hemorrhagic shock, cardiogenic shock, or systemic inflammation.

In one embodiment, a compound of Formula I can be used to treat or prevent an inflammatory skin disease. In one embodiment, the inflammatory skin disease is contact dermatitis, erythema, or psoriasis.

In one embodiment, the inflammatory disease is rheumatoid arthritis. In one embodiment, the inflammatory disease is juvenile idiopathic arthritis, psoriatric arthritis, or osteoarthritis. In another embodiment, the inflammatory disease is an inflammatory disease of the lung, including asthma and chronic obstructive pulmonary disease (COPD); an inflammatory disease of the skin, including psoriasis and contact dermatitis; an inflammatory disease of the intestinal tract, including inflammatory bowel disease, Crohn's disease, and ulcerative colitis; or an inflammatory disease of the liver, including viral hepatitis and autoimmune hepatitis. In one embodiment, the disease is a chronic inflammatory disease such as rheumatoid arthritis. In another embodiment, the inflammatory disease is a disease associated with a chronic inflammatory reaction, such as atherosclerosis or Alzheimer's disease; or with ischemia/reperfusion injury, such as myocardial infarction, stroke or organ transplantation. In one embodiment, the inflammatory disease is an infectious disease such as septic shock.

Therapeutic Administration

In one embodiment, compounds described herein can be formulated into pharmaceutical compositions together with pharmaceutically acceptable carriers for oral administration in solid or liquid form, or for intravenous, intramuscular, subcutaneous, transdermal, or topical administration. In one embodiment, the compound is formulated with a pharmaceutically acceptable carrier for oral administration.

Pharmaceutically acceptable carriers for oral administration include capsules, tablets, pills, powders, troches, and granules. In the case of solid dosage forms, the carrier can comprise at least one inert diluent such as sucrose, lactose or starch. Such carriers can also comprise additional substances other than diluents, e.g., lubricating agents such as magnesium stearate. In the case of capsules, tablets, troches and pills, the carrier can also comprise buffering agents. Carriers, such as tablets, pills and granules, can be prepared with enteric coatings on the surfaces of the tablets, pills or granules. Alternatively, the enteric coated compounds can be pressed into tablets, pills, or granules. Pharmaceutically acceptable carriers include liquid dosage forms for oral administration, e.g., emulsions, solutions, suspensions, syrups and elixirs containing inert diluents commonly used in the art, such as water. Besides such inert diluents, compositions can also include adjuvants, such as wetting agents, emulsifying and suspending agents, and sweetening, flavoring agents.

Pharmaceutically acceptable carriers for topical administration include DMSO (dimethyl sulfoxide), alcohol or propylene glycol that can be employed with patches or other liquid retaining material to hold the medicament in place on the skin. Carriers based on nanoparticles and nanoencapsulates are also convenient for the protection of the active principle and its slow release in the organism or specific tissues.

Pharmaceutically acceptable carriers for intravenous administration include solutions containing pharmaceutically acceptable salts or sugars.

Pharmaceutically acceptable carriers for intramuscular or subcutaneous injection include salts, oils, or sugars.

Carriers such as solvents, water, buffers, alkanols, cyclodextrins and aralkanols can be used. Other auxiliary, non-toxic agents may be included, for example, polyethylene glycols or wetting agents.

Controlled delivery of drugs into the organism is important, especially for drugs that have undesired toxic effects if present systemically or at high local concentrations. CO release can be toxic at high concentrations. For certain applications, a slow release of CO in the blood or in specific target tissues is desirable. Encapsulation within host molecules that are non-toxic is one way to achieve a sustained release of active drugs in the organism. This strategy minimizes the undesired effects that may result from abrupt increases in the concentration and/or availability of a potentially toxic drug.

Cyclodextrins are well known hosts for many drugs and organic molecules and recently have been applied to host organometallic molecules and enhance their delivery through physiological barriers or membranes. In this respect, cyclodextrin has been found to be beneficial for increasing delivery of lipophilic drugs at the skin barrier. (30) Cyclodextrin mediated supramolecular arrangements protect organometallic molecules for prolonged time periods and mask their reactivity, thereby increasing their selectivity towards specific reagents. The hydrophobic part of carbonyl complexes, as those exemplified under Formula I, fit inside β- or γ-cyclodextrin, or similar structures, with the CO groups facing the reaction medium and the organic ligands buried in the cavity. The resulting reduction in reactivity allows for the extension of the range of therapeutic CO-releasing complexes to cationic and anionic ones. Such charged complexes are more reactive and lose CO faster than the neutral ones when unprotected.

Liposomes and other polymeric nanoparticle aggregates are also useful carriers to target the delivery of CO-releasing organometallic complexes and the combined use of cyclodextrins with such aggregates has been considered as a very promising possibility for drug release. (31)

Mesoporous materials are chemically inert three dimensional molecules with infinite arrays of atoms creating channels and cavities of well defined pore size. These molecules are well suited to host organic and organometallic molecules in their pores. In the presence of biological fluids, smaller molecules undergoing acid-base and/or polar interactions with the inner walls of the pores slowly displace the included drugs, resulting in a controlled delivery of the active principle. Such aggregates have been prepared from M41S materials using organometallic molecules. Examples include MCM-41 (linear tubes) and MCM-48 (cavities and pores).

Hosting of compounds of Formula I by cyclodextrin, liposomes, other polymeric nanoparticles, or mesoporous materials can achieve sustained release of CO in vitro.

The pharmaceutically acceptable carriers and compounds described herein can be formulated into unit dosage forms for administration to an animal. The dosage levels of active ingredients (i.e., compounds described herein) in the unit dosage can be varied so as to obtain an amount of active ingredient that is effective to achieve a therapeutic effect in accordance with the desired method of administration. The selected dosage level therefore mainly depends upon the nature of the active ingredient, the route of administration, and the desired duration of treatment. If desired, the unit dosage can be such that the daily requirement for an active compound is in one dose, or divided among multiple doses for administration, e.g., two to four times per day.

In one embodiment, the compounds are administered orally once a day. The compounds described herein generate CO after administration to the body. Although CO is generated preferentially at the sites of inflammation, some of the CO generated will bind to hemoglobin in red blood cells. Thus, dose-finding studies can be guided by measurement of carboxyhemoglobin (COHb) levels in the blood. Methods for the measurement of COHb levels in the blood are known in the art. In normal healthy humans, COHb levels are about 0.5% in healthy nonsmokers and up to 9% in smokers. In one embodiment, the dose level of the compounds described herein is such that no significant rise in COHb levels is observed. However, in some applications, a transient rise in COHb levels up to 10% may be tolerated. This level of COHb is not associated with any symptoms.

In one embodiment, a compound described herein can be administered in a dosage ranging between about 5 mmol/day and about 25 mmol/day, including about 6 mmol/day, about 7 mmol/day, about 8 mmol/day, about 9 mmol/day, about 10 mmol/day, about 11 mmol/day, about 12 mmol/day, about 13 mmol/day, about 14 mmol/day, about 15 mmol/day, about 16 mmol/day, about 17 mmol/day, about 18 mmol/day, about 19 mmol/day, about 20 mmol/day, about 21 mmol/day, about 22 mmol/day, about 23 mmol/day, or about 24 mmol/day, depending on the nature of the CO containing compound and its molar CO content.

In one embodiment, the invention provides the use of a compound of Formula I for the preparation of a medicament for inhibiting tumor necrosis factor production in an animal.

In one embodiment, the invention provides the use of a compound of Formula I for the preparation of a medicament for inhibiting TNF production in a cell.

In one embodiment, the invention provides the use of a compound of Formula I for the preparation of a medicament for treating or preventing an inflammatory disease in an animal.

### EXAMPLES

### Example 1

### Preparation of Compounds I.1-I.2

The general preparation and characterization of compounds of Formula I has been described by Wilkinson, et al. in the references. (28, 29)

Compounds I.1, I.2 and I.6 are described and characterized in E. W. Abel, I. S. Butler and J. G. Reid, J. Chem. Soc., 2068 (1963). (27) We have, however, prepared them according to the modification introduced by Burgmayer and Templeton for the preparation of Compound I.3 (see Example 2). (26) The detailed preparation of Compound I.1 is given.

### Preparation of Compound I.1

A solution containing Mo(CO)₆ was prepared by dissolving 6.60 g (25.00 mmol) and 6.70 g (31.9 mmol) of Et₄NBr in 75 ml of THF. The mixture was refluxed for 2 hours, 30 minutes (Temp.=85-90° C.). Afterwards, the solution was immediately filtered (yellow solution) and half the solvent was evaporated under vacuum. A precipitate started to form and 60 ml of hexane were added to the solution to induce more precipitation. The schlenk tube was kept at −30° C. for 1 hour. After that time, the solution was filtered and the yellow compound obtained was dried in vacuum. Yield: 89%. I.R. (KBr) (νC≡O)(cm⁻¹): 2069 (S), 1912 (S); 1871 (S); S=strong. Elemental Analysis C₁₃H₂₀BrMoNO₅:=446.1496. % experimental (% calculated): C 34.88 (35.00); H 4.82 (4.52); N 3.06 (3.14)

### Preparation of Compound I.2

Compound I.2 was prepared as described above in the preparation of Compound I.1. As will be recognized by those of skill in the art, other compounds described herein can be made similarly using the appropriate tetraalkylammonium halide.

Elemental (C, H, N) analysis confirmed the expected stoichiometry and spectroscopic data (IR, UV/vis, and NMR) were in agreement with those reported in (27) for Compound I.1.

### Example 2

### Preparation of Compound I.3

Compound I.3 was made as described in Burgmayer and Templeton. (26) Mo(CO)₆ (1.50 g; 5.7 mmol) and Et₄NI (1.52 g; 5.9 mmol) were put in a schlenk and 20 ml of THF were added. The suspension was refluxed for 130 minutes. The yellow solution was filtered hot to discard traces of white solid, and then concentrated to half its volume. Hexane was added, and the yellow solid, which precipitated immediately, was filtered and dried under vacuum to yield 2.70 g (96%) of pure compound.

IR (KBr pellet), cm⁻¹: 2072 (m), 1909 (s), 1872 (s).

Elemental Analysis Calculated for C₁₃H₂₀NO₅₁Mo: C, 31.66; H, 4.09; N, 2.84. Found: C, 32.07; H, 3.98; N, 2.85.

### Example 3

### Preparation of Compound I.16

Compounds I.16 and I.17 were prepared according to the procedures described in U.S. Pat. No. 3,278,570 (28), which is incorporated herein by reference. Elemental analysis and IR data were in agreement with those reported in U.S. Pat. No. 3,278,570 and in E. W. Abel, M. A. Bennett and G. Wilkinson, Chemistry and Industry, p. 442 (1960) for Compounds I.16 and I.17.

### Preparation of Compound I.16

A mixture of 3.30 g of Mo(CO)₆ (12.50 mmol) and 2.08 g of KI (12.50 mmol) was heated in 25 ml of freshly distilled diglyme at 120° C. for 1 hour. The yellow suspension was filtered hot and 20 ml of hexane was added to the cooled filtrate. The solid which precipitated was filtered, washed with diethyl ether and dried under vacuum to yield 9.25 g (92%) of K(diglyme)₃ [Mo(CO)₅I]

Elemental Analysis Calculated for C₂₃H₄₂KIMoO₁₄: C, 34.34; H, 5.26. Found: C, 34.14; H, 5.04.

### Example 4

### Preparation of Compound I.17

A mixture of 1.81 g of Mo(CO)₆ (6.91 mmol) and 0.80 g of KBr (6.70 mmol) was heated in 30 ml of freshly distilled diglyme at 120° C. for 2 hours. The yellow suspension was filtered hot and to the cooled filtrate was added 50 ml of hexane. The solid, which precipitated at −30° C., was recrystallized from diglyme/hexane at low temperature to yield a yellow product in the crystalline state (4.42 g; 87%).

Elemental Analysis Calculated for C₂₃BrH₄₂KMoO₁₄: C, 36.47; H, 5.59. Found: C, 36.93; H, 6.35.

IR (KBr pellet), cm⁻¹: 2998 (w, br), 2938-2901 (w, br), 2827 (w, br), 2738 (w, br), 2062 (w), 1917 (s), 1842 (s), 1472 (m), 1454 (m), 1380 (w), 1353 (m), 1288 (w), 1248 (m), 1204 (m), 1113 (s), 1085 (s), 1018 (m), 941 (w), 865 (m), 837 (w), 600 (s), 543 (w).

### Example 5

### Preparation of Compound I.18

A mixture of 1.81 g of Mo(CO)₆ (6.91 mmol) and 0.97 g of NH₄I (6.70 mmol) was heated in 30 ml of freshly distilled diglyme at 120° C. for 1 hour. The yellow suspension was filtered hot and 50 ml of hexane was added to the cooled filtrate. The yellow solid which precipitated was recrystallized from diethyl ether/hexane and left at −30° C. After filtering and drying under vacuum [NH₄](diglyme)₃[Mo(CO)₅I] was obtained in quantitative yield (4.46 g).

Analysis Calculated for C₁₁H₁₈₁MoNO₈: C, 35.26; H, 5.90; N, 1.78. Found: C, 35.70; H, 5.75; N, 1.80.

IR (KBr pellet), cm⁻¹: 3493 (w, br), 3082 (vw, br), 2993 (w), 2896 (m), 2828 (m), 2747 (vw), 2737 (vw), 2059 (w), 1919 (s), 1850 (s), 1471 (m), 1454 (m), 1428 (m), 1378 (w), 1352 (m), 1288 (w), 1247 (m), 1204 (m), 1112 (s), 1086 (s), 1017 (m), 940 (w), 862 (s), 837 (w), 836 (m), 607 (s), 597 (s), 541 (w).

### Example 6

### Preparation of Compound I.19

A mixture of 0.27 g of Mo(CO)₆ (1.03 mmol), 0.12 g of KBr (1.00 mmol) and 0.31 g of benzo-18-crown-6-ether (1.00 mmol) was refluxed in 15 ml of freshly distilled THF for 2 hours. The yellow solution was filtered and a brown oil formed and started to separate. After decanting the oil, 40 ml of hexane were added and the schlenk was left at 0° C. to isolate a yellow solid. Extraction with CH₂Cl₂ afforded K[benzo-18-crown-6-ether] [Mo(CO)₅Br] as yellow needles (75% yield, 0.5 g).

Analysis Calculated for BrC₂₁H₂₄KMo₁₁: C, 37.80; H, 3.62. Found: C, 37.25; H, 3.83.

IR (KBr pellet), cm⁻¹: 3069 (w), 2955 (m), 2941 (m), 2921 (m), 2067 (m), 1977 (w), 1932 (sh, s), 1914 (s), 1842 (s), 1834 (s), 1638 (br, w), 1596 (w), 1523 (w), 1505 (s), 1478 (w), 1457 (m), 1436 (w), 1382 (w), 1355 (m), 1343 (m), 1325 (m), 1286 (m), 1247 (s), 1214 (s), 1178 (br, sh, w), 1166 (w), 1126 (m), 1116 (m), 1108 (m), 1097 (sh, w), 1083 (w), 1075 (w), 1055 (w), 961 (m), 952 (m), 935 (w), 922 (w), 908 (w), 882 (w), 860 (w), 848 (w), 833 (w), 806 (br, w), 731 (m), 737 (s), 598 (s), 542 (m), 507 (w), 463 (w).

¹H NMR (CD₂Cl₂, 300 MHz): 7.00-6.90 (c, 4H, C₆H₄ crown ether), 4.22+3.95+3.77−3.42 (c, 10H, aliphatic crown ether protons).

### Example 7

### Spontaneous CO release

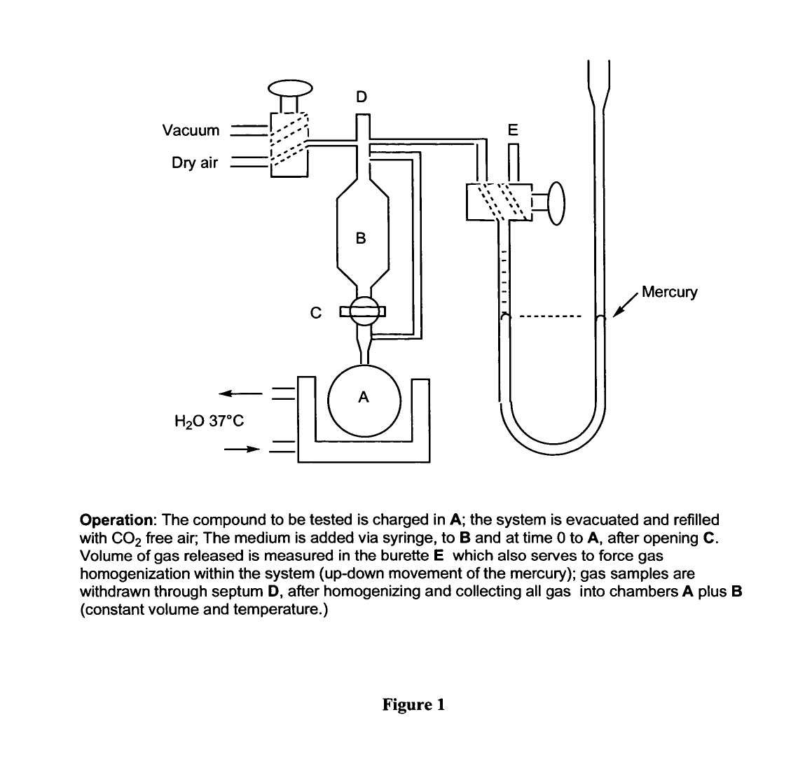

These studies were conducted in the apparatus shown in FIG. 1. CO detection was carried out by Gas Chromatography using a thermal conductivity detector (TCD) for the quantification of CO and CO₂. The experiments were done under an initial atmosphere of reconstituted air, free of CO and CO₂. The medium used was RPMI with 10% Fetal Bovine Serum. The suspension of Compound I.1 in RPMI/FBS or water was magnetically stirred and its temperature was kept at 37° C. by using a thermostated circulating bath. Samples were withdrawn with gas-tight Hamilton syringes after homogenization of the head-space at given time intervals, preferably 2 hours, 4 hours and 6 hours. No attempts were made to quantify the CO gas remaining dissolved which, at this temperature, is very small due to the very low solubility of CO and the small total volume of solution used (3 mL). The volumes of CO released are usually in the range between 0.5-3 mL. Due to the low solubility of Compound I.1 in water and RPMI, the CO release experiments were carried out on suspensions with the following amounts of Compound I.1: 2.4-3.5 mg Compound I.1/ml RPMI; 5.9 mg Compound I.1/ml H₂O (pH=2.13); 5.8 mg Compound I.1/ml H₂O (pH=8.3); 4.6 mg Compound I.1/ml olive oil. The amount of CO released (in equivalents of CO) is given in Table 1.

TABLE 1
Equivalents of CO released from suspensions of
compounds of Formula I in different media, at 37° C.
in the dark, (numbers are averages)

| | Time of | | H₂O | H₂O | Olive |
|---|---|---|---|---|---|
| Compound | reaction | RPMI | pH = 2.13 | pH = 8.3 | oil |
| Compound I.1 | 2 hours | 1.82 | 0.98 | 1.24 | 0.08 |
| | 4 hours | 2.16 | 1.00 | 1.03 | 0.25 |
| | 6 hours | 2.27 | 0.93 | 0.98 | 0.53 |
| Compound I.6 | 2 hours | 0.42 | Not | Not | Not |
| | 4 hours | 1.00 | tested | tested | tested |
| | 6 hours | 1.25 | | | |
| Compound I.16 | 2 hours | 0.94 | Not | Not | Not |
| | 4 hours | 1.20 | tested | tested | tested |
| | 6 hours | 1.28 | | | |
| Compound I.18 | 2 hours | 0.55 | Not | Not | Not |
| | 4 hours | 0.73 | tested | tested | tested |
| | 6 hours | 0.79 | | | |

As a possible result of the use of suspensions, the number of CO equivalents released in RPMI varied slightly. As an example of the possible variations, the average of eight independent assays done with suspensions of Compound I.1 is given in Table 2.

TABLE 2
Equivalents of CO released by Compound I.1 in suspension in RPMI at
37° C. in the dark. Average from eight independent assays.

| | CO equivalents |
|---|---|
| Time/hours | (average ± standard deviation) |
| 0.5 | 0.64 ± 0.11 |
| 1 | 1.56 ± 0.13 |
| 2 | 1.82 ± 0.01 |
| 3 | 2.42 ± 0.38 |
| 4 | 2.16 ± 0.13 |
| 5 | 2.43 ± 0.39 |
| 6 | 2.27 ± 0.21 |
| 7 | 2.51 ± 0.00 |
| 24 | 2.40 ± 0.00 |

### Example 8

### CO Release in the Presence of Reactive Oxygen Species (“ROS”) (e.g., Hydrogen Peroxide (H₂O₂), tert-Butyl Hydroperoxide (t-BuOOH; TBHP) and Potassium Superoxide (KO₂))

The studies were done using the same method and apparatus described in Example 7 with the following modifications: RPMI/FBS was replaced by double distilled water in the experiments with H₂O₂ and TBHP and by tetrahydrofuran (THF) for the experiments with KO₂; the temperature was kept at 25° C. The concentration of Compound I.1 was approximately 1 mM and the ration of concentrations of H₂O₂, TBHP and KO₂ relative to Compound I.1 was 100:1. The amount of CO₂ generated was also measured in the same experiment to ascertain the concurrent oxidation of coordinated CO. TBHP was added from a 70% aqueous solution and H₂O₂ from a 30% aqueous solution. The results are given in Table 3.

TABLE 3
Equivalents of CO and CO₂ released with different
ROS at 25° C. in the dark.

| | Time of | TBHP | | H₂O₂ | | KO₂ | |
|---|---|---|---|---|---|---|---|
| Compound | reaction | CO | CO₂ | CO | CO₂ | CO | CO₂ |
| Compound | 1 h | 2.51 | 0.51 | 1.08 | 0.41 | 1.94 | 0.00 |
| I.1 | 3 h | 3.77 | 0.95 | 1.49 | 0.80 | 3.22 | 0.00 |
| | 5 h | 3.94 | 1.07 | 1.46 | 0.90 | 2.54 | 0.00 |
| | 24 h | 3.93 | 1.13 | 1.43 | 0.89 | 2.29 | 0.00 |
| Compound | 1 h | 0.48 | 0.00 | 1.31 | 0.14 | Not | Not |
| I.6 | 3 h | 1.75 | 0.24 | 3.04 | 0.44 | Tested | Tested |
| | 5 h | 3.51 | 0.55 | 3.20 | 0.51 | | |
| | 24 h | 4.29 | 0.99 | 1.91 | 0.35 | | |

### Example 9

### Toxicity In Vitro

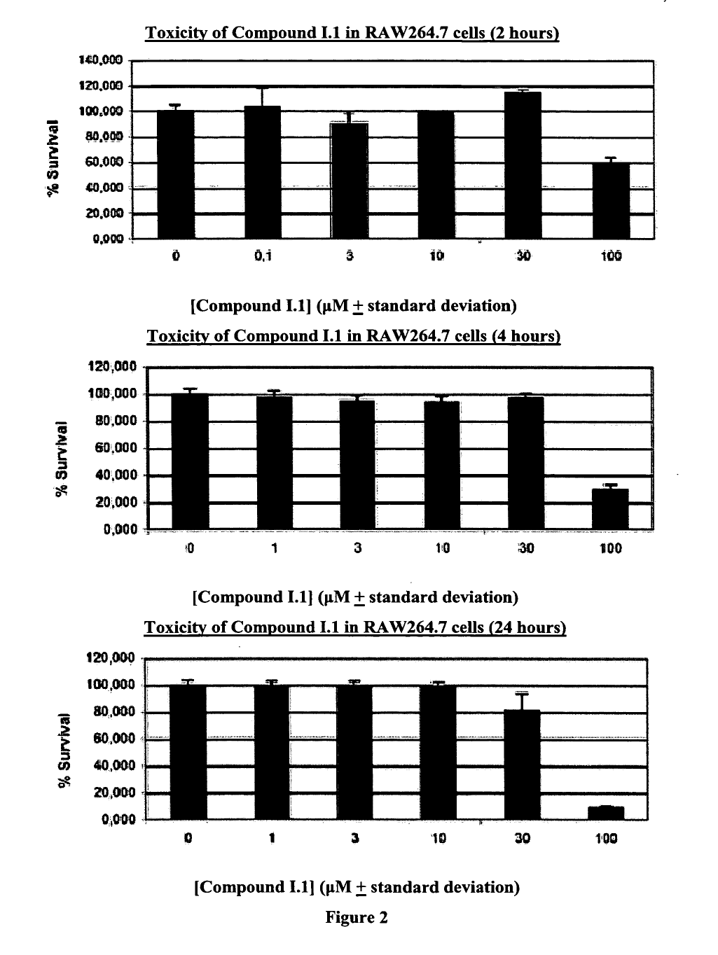

The cell toxicity of Compound I.1 was tested with RAW264.7 cells using the MTT assay to ascertain cell viability. Cells were seeded at 10⁵ per well with different concentrations of Compound I.1 and incubated for two to 24 hours; cell viability was then determined by the MTT assay; cells were incubated for 1 hour with 1 mg/ml MTT in DMEM, the supernatant was discarded and formazan crystals were dissolved in 150 ml DMSO. The results are given in FIG. 2 for 2, 4 and 24 hours of incubation.

### Example 10

### Toxicity In Vivo

Compound I.1 was dissolved in olive oil and administered to Sprague Dawley rats at a daily dose of 80 mg/kg for 20 days. At the end of the treatment the rats were anesthetized, blood was collected and organ samples were fixed in formalin for histological analysis. No signs of liver or kidney toxicity were observed. The serum values for glutamic oxalacetic transaminase (sGOT), glutamic pyruvic transaminase (sGPT), creatinine and urea were in the normal range. Histologic analysis did not reveal any gross alterations in the liver, kidney, heart, and spleen.

### Example 11

### CO Release In Vivo

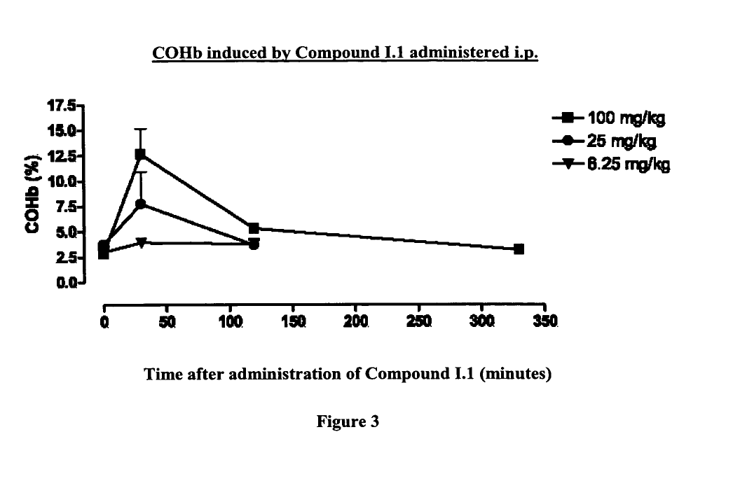

Nine week old Balb/c mice with a body weight of about 20 g were injected by the intraperitoneal route with Compound I.1 dissolved in a propylene glycol-water mixture. Three doses (100, 25 and 6.25 mg/kg) were used. At various times after the administration of the Compound I.1 blood was collected and CO-hemoglobin levels were determined using an oximeter. The results were obtained after 0, 30, 120 and, in one case, 330 minutes are given in FIG. 3. The results show an increase in CO levels during the first time interval, followed by a slow decline from peak CO-levels over the next few time intervals.

### Example 12

### Inhibition of LPS-Induced TNF Production in Mice

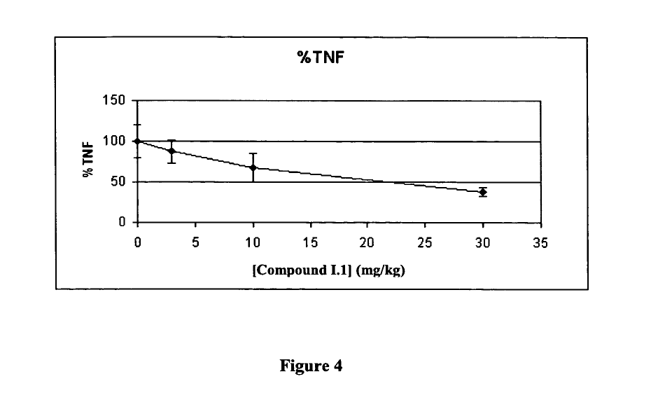

The ability of Compound I.1 to inhibit TNF production was tested in mice according to the procedure of WO 98/38179. Eight week old, female Balb/c mice received intraperitoneal injections of Compound I.1 at different doses (3, 10 and 30 mg/kg) or vehicle (carboxymethylcellulose 0.5%, Tween80 0.5%) only. Thirty minutes later all mice received intraperitoneal injections of LPS 0111:B4 Sigma at a dose of 0.3 mg/kg. Ninety minutes after the injection of LPS, serum samples were collected and analyzed for TNF content by ELISA. The data are shown in FIG. 4. These data show that Compound I.1 inhibited TNF production with an ED₅₀ of about 22 mg/kg.

### Example 13

### Impact on Mortality in Mice After Injection of a Lethal Dose of LPS

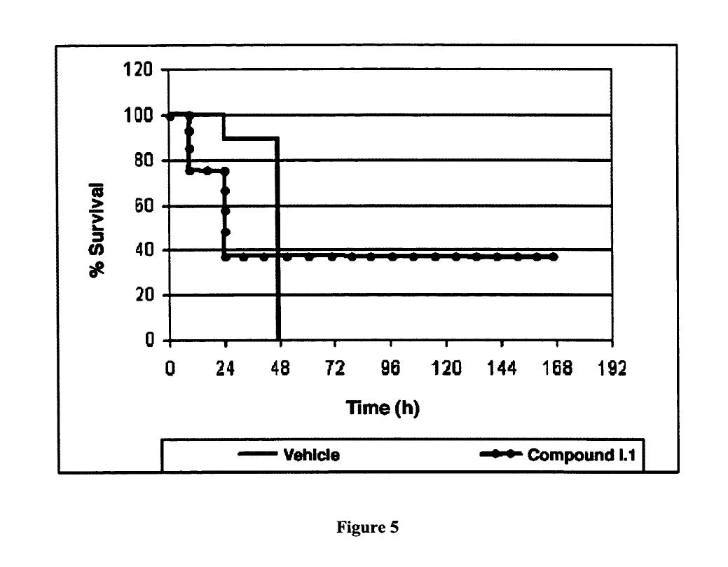

Seventeen eight week old Balb/c mice received one intraperitoneal injection of LPS at a dose of 10 mg/kg at time zero. One group of eight mice received four intraperitoneal injections of Compound I.1, each at a dose of 20 mg/kg, at 60 and 30 minutes before LPS and at 4 hours and 9 hours after LPS. A second group of 9 mice received four intraperitoneal injections of vehicle (carboxymethylcellulose 0.5%, Tween80 0.5%) at 60 and 30 minutes before LPS and at 4 hrs and 9 hrs after LPS. Survival of the mice was monitored for 168 hours. As shown in FIG. 5, all nine vehicle treated mice were dead at 47 hours following LPS injection while three of the eight mice treated with Compound I.1 remained alive at 168 hours following LPS injection, at which time they were sacrificed. These data demonstrate a significant inhibition of LPS-induced lethal effects of lipopolysaccharide by Compound I.1.

### Example 14

### Treatment of Adjuvant Arthritis in Rats with Compound I.1

Adjuvant arthritis was induced in 11 week old, outbred Wistar rats (376-400 g) by a single intradermal injection into the subplanatar area of the right hind paw of 100 microliter of a 10 mg/ml suspension of mycobacterium butyricum in incomplete Freund's Adjuvant. The disease was induced in 3 groups of rats each consisting of 7 animals. Group 1 (control) did not receive any treatment. Groups 2 and 3 received daily applications of methylene chloride (positive control) (500 mg/kg), or Compound I.1 (80 mg/kg), respectively. Both compounds were administered in olive oil by oral gavage. Treatment was initiated at day 10 after disease induction when signs of arthritis began to appear in the injected footpad as well as in the contralateral footpad. The treatment lasted for 20 days until day 29 after disease induction. At day 20 of treatment, the control group was reduced by three rats with severe arthritis. These three rats were then treated with Compound I.1 for 10 days. All animals were evaluated daily by determination of body weight, foot pad volume (performed by a water displacement method using a plethysmometer, Ugo Basile, Comerio, Italy), ankle circumference (using a flexible measuring tape) and arthritic index that is based on levels of erythema and oedema of the entire paws and digits, number of joints involved, spondilosis, lesions on tail, movement capacity and infections (0=normal, 1=swelling and/or redness of injected paw; 4=severe arthritis of the entire injected paw and digits; +2=2 joints are involved; +3=>2 joints are involved; +1=infection of paws; +1=tail lesions; +1=movement incapacity; +1=spondilosis). The sum of the parameters is calculated as an arthritis index with a maximum possible score of 11.

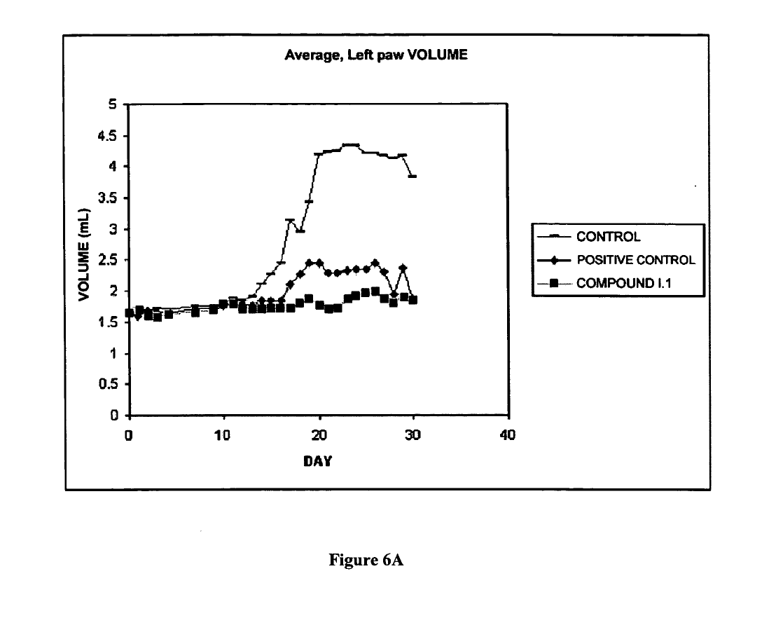

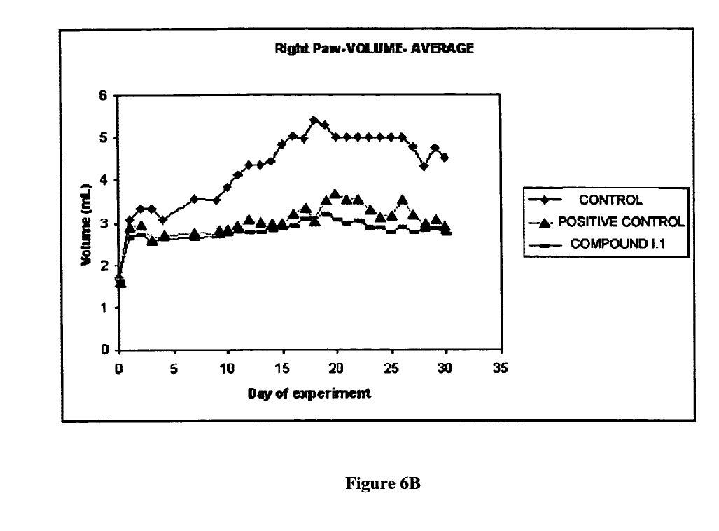

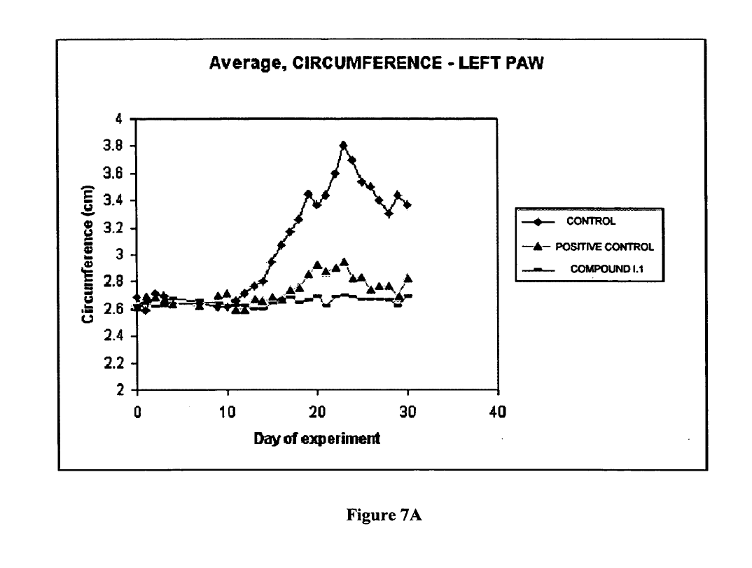

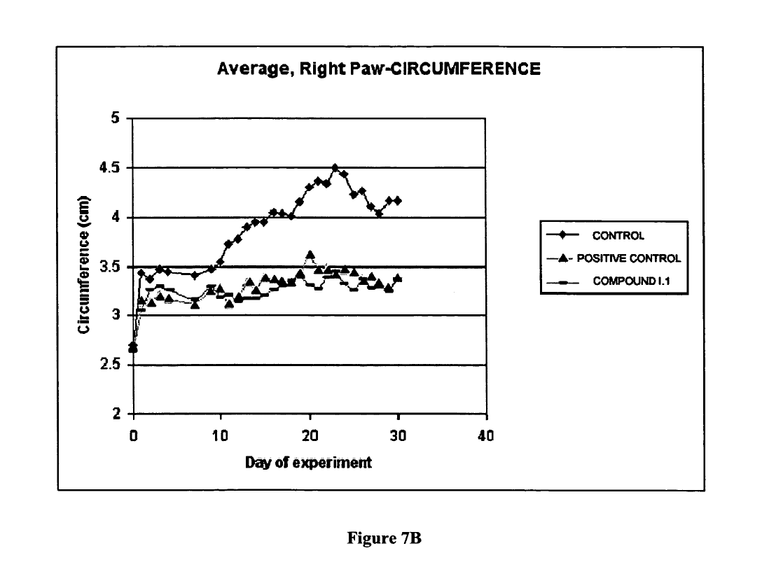

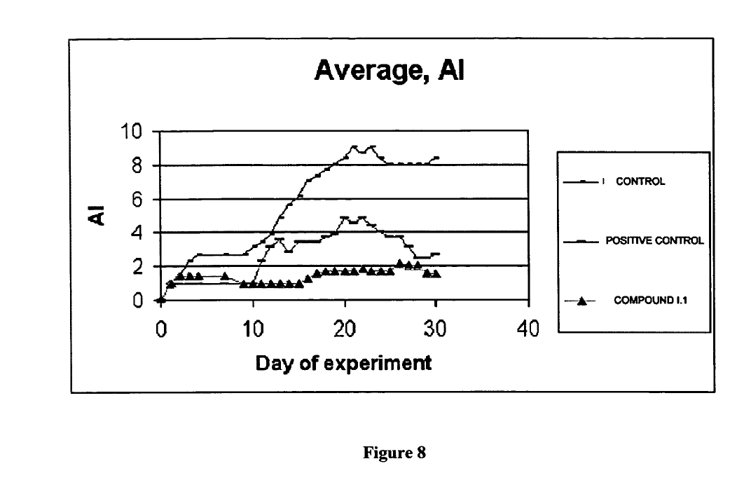

The results are shown in FIGS. 6, 7 and 8. FIGS. 6A-6B show the average left (FIG. 6A) or right (FIG. 6B) paw volume in rats of the control, positive control-treated and Compound I.1-treated groups. FIGS. 7A-7B show the average left (FIG. 7A) or right (FIG. 7B) paw circumference in rats of the control, positive control-treated and Compound I.1-treated groups. FIG. 8 demonstrates the arthritis index in rats of the control, positive control-treated and Compound I.1-treated groups. Methylene chloride was used as a positive control in each instance. Methylene chloride generates CO when it is metabolized in the liver and has previously been shown to have beneficial effects in a rat arthritis model (US 2003/0068387). Compound I.1 at 80 mg/kg was superior to methylene chloride at 500 mg/kg in all measured parameters. The three rats of the control group that were treated with Compound I.1 from day 20 on showed also signs of improvements after 10 days.

### Example 15

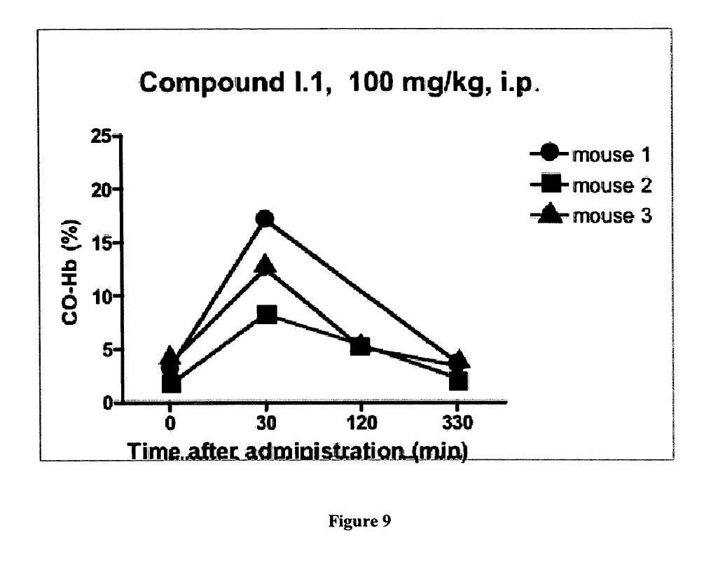

Compound I.1 was administered intraperitonally to mice at a concentration of 100 mg/kg using propylene glycol/water ca.˜2:1 as vehicle. The amount of COHb (carboxyhemoglobin) was monitored with an oximeter in blood samples withdrawn at 0, 30, 120 and 330 minutes after administration. The results are shown in FIG. 9 and show a peaked level of CO after 30 minutes followed by a slow decline.

### Example 16

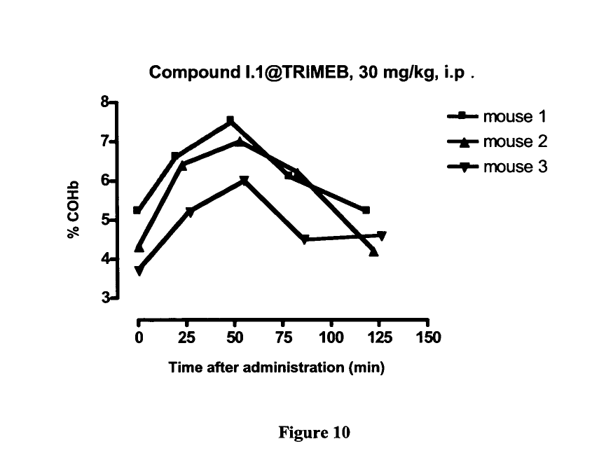

Compound I.1 was encapsulated in methylated β-cyclodextrin, 2,3,6-tri-O-methyl-β-cyclodextrin, known in the art as TRIMEB, by a standard technique. The encapsulated Compound I.1@TRIMEB (“@” means “encapsulated in”) was administered intraperitonally to mice at a concentration of 30 mg/kg using phosphate buffered saline (PBS) as vehicle. The amount of COHb (carboxyhemoglobin) was monitored with an oximeter in blood samples withdrawn after 30, 60, 90 and 120 minutes after administration. The results are shown in FIG. 10 and demonstrate a less intensive and slower release of CO in the encapsulated complexes with a more sustained profile.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

### REFERENCES

1. Campbell, I. K., L. J. Roberts, and I. P. Wicks. 2003. Molecular targets in immune-mediated diseases: the case of tumour necrosis factor and rheumatoid arthritis. Immunol Cell Biol 81:354-366.
2. Lovell, D. 2004. Biologic agents for the treatment of juvenile rheumatoid arthritis: current status. Paediatr Drugs 6:137-146.
3. Peloso, P. M., and J. Braun. 2004. Expanding the armamentarium for the spondyloarthropathies. Arthritis Res Ther 6 Suppl 2:S36-43.
4. Sandborn, W. J. 2003. Strategies for targeting tumour necrosis factor in IBD. Best Pract Res Clin Gastroenterol 17:105-117.
5. Tilg, H., and A. Kaser. 2002. Antitumour necrosis factor therapy in Crohn's disease. Expert Opin Biol Ther 2:715-721.
6. Krueger, G., and K. Callis. 2004. Potential of tumor necrosis factor inhibitors in psoriasis and psoriatic arthritis. Arch Dermatol 140:218-225.
7. Mikuls, T. R., and L. W. Moreland. 2003. Benefit-risk assessment of infliximab in the treatment of rheumatoid arthritis. Drug Saf 26:23-32.
8. Feldmann, M., and R. N. Maini. 2001. Anti-TNF alpha therapy of rheumatoid arthritis: what have we learned? Annu Rev Immunol 19:163-196.
9. Otterbein, L. E. 2002. Carbon Monoxide: Innovative Anti-inflammatory Properties of an Age-Old Gas Molecule. Antioxid Redox Signal 4:309-319.
10. Ryter, S. W., and L. E. Otterbein. 2004. Carbon monoxide in biology and medicine. Bioessays 26:270-280.
11. Otterbein, L. E., F. H. Bach, J. Alam, M. Soares, H. Tao Lu, M. Wysk, R. J. Davis, R. A. Flavell, and A. M. Choi. 2000. Carbon monoxide has anti-inflammatory effects involving the mitogen-activated protein kinase pathway. Nat Med 6:422-428.
12. Morse, D., S. E. Pischke, Z. Zhou, R. J. Davis, R. A. Flavell, T. Loop, S. L. Otterbein, L. E. Otterbein, and A. M. Choi. 2003. Suppression of inflammatory cytokine production by carbon monoxide involves the JNK pathway and AP-1. J Biol Chem 278:36993-36998.
13. Sarady, J. K., B. S. Zuckerbraun, M. Bilban, O. Wagner, A. Usheva, F. Liu, E. Ifedigbo, R. Zamora, A. M. Choi, and L. E. Otterbein. 2004. Carbon monoxide protection against endotoxic shock involves reciprocal effects on iNOS in the lung and liver. Faseb J. 18:854-856.
14. Ndisang, J. F., P. Gai, L. Berni, C. Mirabella, R. Baronti, P. F. Mannaioni, and E. Masini. 1999. Modulation of the immunological response of guinea pig mast cells by carbon monoxide. Immunopharmacology 43:65-73.
15. Song, R., R. S. Mahidhara, Z. Zhou, R. A. Hoffman, D. W. Seol, R. A. Flavell, T. R. Billiar, L. E. Otterbein, and A. M. Choi. 2004. Carbon monoxide inhibits T lymphocyte proliferation via caspase-dependent pathway. J Immunol 172:1220-1226.
16. Sawle, P., R. Foresti, B. E. Mann, T. R. Johnson, C. J. Green, and R. Motterlini. 2005. Carbon monoxide-releasing molecules (CO-RMs) attenuate the inflammatory response elicited by lipopolysaccharide in RAW264.7 murine macrophages. Br J Pharmacol. 145(6):800-10.
17. Lee, T. S., and L. Y. Chau. 2002. Heme oxygenase-1 mediates the anti-inflammatory effect of interleukin-10 in mice. Nat Med 8:240-246.
18. Otterbein, L. E., M. P. Soares, K. Yamashita, and F. H. Bach. 2003. Heme oxygenase-1: unleashing the protective properties of heme. Trends Immunol 24:449-455.
19. Motterlini, R., J. E. Clark, R. Foresti, P. Sarathchandra, B. E. Mann, and C. J. Green. 2002. Carbon monoxide-releasing molecules: characterization of biochemical and vascular activities. Circ Res 90:E17-24.
20. Johnson, T. R., B. E. Mann, J. E. Clark, R. Foresti, C. J. Green, and R. Motterlini. 2003. Metal carbonyls: a new class of pharmaceuticals? Angew Chem Int Ed Engl 42:3722-3729.
21. Guo, Y., A. B. Stein, W. J. Wu, W. Tan, X. Zhu, Q. H. Li, B. Dawn, R. Motterlini, and R. Bolli. 2004. Administration of a CO-Releasing Molecule at the Time of Reperfusion Reduces Infarct Size In Vivo. Am J Physiol Heart Circ Physiol. 286(5):H1649-53.
22. Fischer, E. O., and K. Ofele. 1959. Methylpyridin-Chrom(O)-Tricarbonyl. Zeitschrift Fur Naturforschung Part B-Chemie Biochemie Biophysik Biologie Und Verwandten Gebiete 14:736-737.
23. Fischer, E. O., and K. Ofele. 1960. Uber Aromatenkomplexe Von Metallen 0.37. Zur Aromatenkomplexbildung Des Pyridins Mit Chromhexacarbonyl. Chemische Berichte-Recueil 93:1156-1161.
24. Douglas, W., and J. K. Ruff. 1974. Preparation of Some Group Vi Fluorometal Carbonyl Derivatives. Journal of Organometallic Chemistry 65:65-69.
25. Cihonski, J. L., and R. A. Levenson. 1975. Crown Ethers in Inorganic-Chemistry—Preparation and Characterization of Group 6 Pentacarbonyl Hydroxides and Fluorides. Inorganic Chemistry 14:1717-1720.
26. Burgmayer, S. J. N., and J. L. Templeton. 1985. Synthesis and Structure of a 7-Coordinate Molybdenum Carbonyl Fluoride Derivative—Et4n Mo(Co)2(S2cnet2)2f. Inorganic Chemistry 24:2224-2230.
27. Abel, E. W., J. G. Reid, and I. S. Butler. 1963. Anionic Halogenopentacarbonyls of Chromium, Molybdenum, and Tungsten. Journal of the Chemical Society 2068.
28. U.S. Pat. No. 3,278,570.
29. Abel, E. W., Bennett, M. A., Wilkinson, G. 1960. Chem. and Ind. 442.
30. T. Loftsson, M. Masson, Int. 2001. J. Pharm. 225:15.
31. D. Duchene, G. Ponchel, D. Wouessidj ewe. 1999. Adv. Drug Delivery Rev. 36, 29.

We claim:

1. A method for inhibiting tumor necrosis factor (TNF) production in an animal in need thereof, comprising administering to the animal an effective amount of a compound of the Formula I:
[Mo(CO)₅Y]Q I
wherein
Y is bromide, chloride or iodide;
Q is [NR₄]⁺, complexed with one cyclic polyether molecule or one or more acyclic polyether molecules, or
[NH₄]⁺Na⁺, K⁺, Mg²⁺, C²⁺, or Zn²⁺, wherein each is free or complexed with one cyclic polyether molecule or one or more acyclic polyether molecules; and
wherein
each R is independently alkyl.

2. The method of claim 1, wherein the cyclic polyether molecule is an 18-crown-6 ether or a 15-crown-5 ether.

3. The method of claim 1, wherein the one or more acyclic polyether molecules are of the formula R¹O(CH₂CH₂O)ₙR₂ wherein n is greater than or equal to 1, R¹ and R² are each independently H or alkyl, and each polyether molecule is the same or different.

4. The method of claim 3, wherein n is not greater than 200.

5. The method of claim 3, wherein n is not greater than 100.

6. The method of claim 3, wherein n is within 1 to 75 or 1 to 50.

7. A method for inhibiting TNF production in a cell, comprising contacting the cell with a compound of the Formula I:
[Mo(CO)₅Y]Q I
wherein
Y is bromide, chloride or iodide; and
Q is [NR₄]⁺, complexed with one cyclic polyether molecule or one or more acyclic polyether molecules, or
[NH₄]⁺Na⁺, K⁺, Mg²⁺, C²⁺, or Zn²⁺, wherein each is free or complexed with one cyclic polyether molecule or one or more acyclic polyether molecules; and
wherein
each R is independently alkyl.

8. The method of claim 7, wherein the cyclic polyether molecule is an 18-crown-6 ether or a 15-crown-5 ether.

9. The method of claim 7, wherein the one or more acyclic polyether molecules are of the formula R¹O(CH₂CH₂O)ₙR wherein n is greater than or equal to 1, R¹ and R² are each independently H or alkyl, and each polyether molecule is the same or different.

10. The method of claim 9, wherein n is not greater than 200.

11. The method of claim 9, wherein n is not greater than 100.

12. The method of claim 9, wherein n is within 1 to 75 or 1 to 50.

13. A method for treating or preventing an inflammatory disease in an animal in need thereof, comprising administering to the animal an effective amount of a compound of the Formula I:
[Mo(CO)₅Y]Q I
wherein
Y is bromide, chloride or iodide; and
Q is [NR₄]⁺, complexed with one cyclic polyether molecule or one or more acyclic polyether molecules, or
[NH₄]⁺Na⁺, K⁺, Mg²⁺, Ca²⁺, or Zn²⁺, wherein each is free or complexed with one cyclic polyether molecule or one or more acyclic polyether molecules; and
wherein
each R is independently alkyl.

14. The method of claim 13, wherein the cyclic polyether molecule is an 18-crown-6 ether or a 15-crown-5 ether.

15. The method of claim 13, wherein the one or more acyclic polyethers are of the formula R¹O(CH₂CH₂O)ₙR² wherein n is greater than or equal to 1, R¹ and R² are each independently H or alkyl, and each polyether molecule is the same or different.

16. The method of claim 15, wherein n is not greater than 200.

17. The method of claim 15, wherein n is not greater than 100.

18. The method of claim 15, wherein n is within 1 to 75 or 1 to 50.

19. The method of claim 13, wherein Q is complexed with from 1 to 12 polyether molecules.

20. The method of claim 13, wherein Q is complexed with a polyether molecule from the 18-crown-6 family or the 15-crown-5 family.

21. The method of claim 20, wherein the polyether molecule is [18]-crown-6, [15]-crown-5, dibenzo[18]-crown-6, or dicyclohexyl[18]-crown-6.

22. The method of claim 20, wherein Q is K⁺ or NH₄⁺ complexed with a polyether molecule from the 18-crown-6 family.

23. The method of claim 22, wherein Q is K⁺ or NH₄⁺ complexed with dibenzo[18]-crown-6 or dicyclohexyl[18]-crown-6.

24. The method of claim 20, wherein Q is Na⁺ complexed with a polyether molecule from the 15-crown-6 family.

25. The method of claim 24, wherein Q is Na⁺ complexed with [15]-crown-5.

26. The method of claim 13, wherein the polyether molecule is monoglyme, diglyme, triglyme, PEG 400, PEG 1000, PEG 2000, PEG 3000 and PEG 4000, or methylPEG400.

27. The method of claim 13, wherein Q is K⁺ or [NH₄]⁺ complexed with three diglymes or three diethers.

28. The method of claim 13, wherein the compound is one of the following compounds:

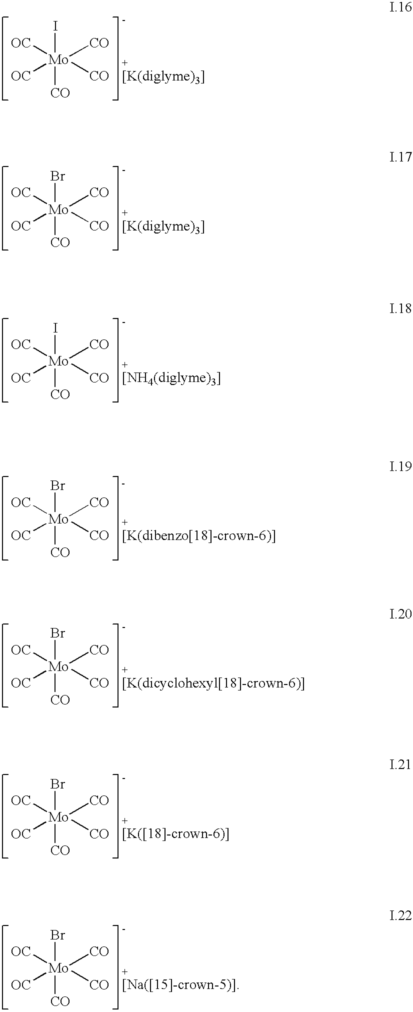

29. The method of claim 13, wherein the inflammatory disease is arthritis.

30. The method of claim 13, wherein the inflammatory disease is rheumatoid arthritis.

31. The method of claim 13, wherein the inflammatory disease is juvenile idiopathic arthritis, psoriatric arthritis, or osteoarthritis.

32. The method of claim 13, wherein the inflammatory disease is asthma, chronic obstructive pulmonary disease, or an inflammatory lung disease.

33. The method of claim 13, wherein the inflammatory disease is ulcerative colitis, Crohn's disease, or an inflammatory bowel disease.

34. The method of claim 13, wherein the inflammatory disease is a disease associated with a chronic inflammatory reaction.

35. The method of claim 13, wherein the inflammatory disease is atherosclerosis or Alzheimer's disease.

36. The method of claim 13, wherein the inflammatory disease is psoriasis, contact dermatitis, or an inflammatory skin disease.

37. The method of claim 13, wherein the inflammatory disease is a disease associated with ischemia/reperfusion injury.

38. The method of claim 13, wherein the inflammatory disease is myocardial infarction, stroke, or organ transplantation.

39. The method of claim 13, wherein the inflammatory disease is viral hepatitis, autoimmune hepatitis, or an inflammatory disease of the liver.

40. The method of claim 13, wherein the inflammatory disease is septic shock or an infectious disease.

41. A method for identifying a compound that inhibits TNF production comprising the steps of
(a) contacting a test cell with a compound of Formula I:
[Mo(CO)₅Y]Q I
wherein
Y is bromide, chloride or iodide; and
Q is [NR⁴]⁺, complexed with one cyclic polyether molecule or one or more acyclic polyether molecules, or
[NH₄]⁺Na⁺, K⁺, Mg²⁺, Ca²⁺, or Zn²⁺, wherein each is free or complexed with one cyclic polyether molecule or one or more acyclic polyether molecules; and
wherein
each R is independently alkyl;
(b) determining a level of TNF produced in a test cell sample isolated from the test cell;
(c) comparing the level of TNF produced in the test cell sample to a level of TNF produced in a control cell sample isolated from a control cell that has not been contacted with the compound of Formula I.

42. The method of claim 41, wherein a compound of Formula I that inhibits TNF production is identified when the level of TNF produced in the test cell sample is less than the level of TNF produced in the control cell sample.

43. A method for identifying compounds that inhibit TNF production in an animal comprising the steps of
(a) administering to an animal a compound of Formula I:
[Mo(CO)₅Y]Q I
wherein
Y is bromide, chloride or iodide; and
Q is [NR₄]⁺, complexed with one cyclic polyether molecule or one or more acyclic polyether molecules, or
[NH₄]⁺Na⁺, K⁺, Mg²⁺, Ca²⁺, or Zn²⁺, wherein each is free or complexed with one cyclic polyether molecule or one or more acyclic polyether molecules; and
wherein
each R is independently alkyl;
(b) determining a level of TNF produced in the animal;
(c) comparing the level of TNF produced in the animal to a level of TNF produced in a control animal that has not been administered the compound of Formula I.

44. The method of claim 43, wherein a compound of Formula I that inhibits TNF production is identified when the level of produced TNF in the animal is less than the level of produced TNF in the control animal.
